(19) United States
(12) Patent Application Publication
(10) Pub. No.: US 2008/0202960 A1
(43) Pub. Date: Aug. 28, 2008

(54) Disposable toothbrush cover and method of using

(76) Inventor: Anne Donohue, North Shore, CA (US)

Correspondence Address: Ms. Anne Donohue;c/o Warren E. Small, Esq., Post Office Box 1650, Pebble Beach, CA, 93953-1650, US

(21) Appl. No.: 12/011,945

(22) Filed: Jan. 30, 2008

Related U.S. Application Data
(60) Provisional application No. 60/903,092, filed on Feb. 23, 2007.

(51) Int. Cl. B65D 83/00; B65D 65/02; B65D 81/26; B65B 1/04; B65D 81/24

(52) U.S. Cl. 206/362.3; 150/154; 206/204; 206/209.1; 53/469

(57) ABSTRACT

An apparatus and method for sanitizing the bristles of a toothbrush before and between uses by wrapping the bristles or the entire toothbrush in a leak-proof and disposable cover that has been impregnated with a sanitizing agent.

### FEDERALLY SPONSORED RESEARCH

Not Applicable

### SEQUENCE LISTING OR PROGRAM

Not Applicable

### FIELD OF THE INVENTION

The present invention generally relates to the field of toothbrush sanitizing devices for use in the home or while traveling. More particularly, the present invention relates to the field of toothbrush sanitizers which sanitize toothbrush bristles before and between uses by enveloping the toothbrush bristles in a leak-proof, disposable pouch or cover that has been impregnated with a sterilizing or sanitizing agent.

### BACKGROUND OF THE INVENTION—DESCRIPTION OF THE PRIOR ART

The prior art and oral hygiene professionals recognize that, between uses, toothbrushes are susceptible to contamination by germs, bacteria, and other undesirable foreign objects such as dust particles and other such airborne debris. These contaminants are subsequently introduced into the mouth when the user next uses the toothbrush thereby presenting a risk to the oral hygiene and overall health of the user. Further, if the user is or has been ill, that user's toothbrush can reinfect the user with the same germs that caused the illness to begin with. Further still, even if the user has developed an immunity to the illness, that user's toothbrush can contaminate other surfaces and infect (a) other people in the user's household or office, or (b) other people who use the same facility where the user engaged in brushing teeth. The prior art contains numerous attempts to solve the foregoing problems by disclosing numerous embodiments of toothbrush sanitizing devices.

Examples of such configurations include U.S. Pat. No. 4,915,219 issued to Ottimo (1990) and U.S. Pat. No. 4,997,629 issued to Marchand et. al (1991) which recognized the economic benefit of sterilizing toothbrushes between uses rather than simply disposing of the toothbrushes once they've been used. Several inventors have addressed the foregoing problem through the use of sterilization containers. U.S. Pat. No. 1,194,540 issued to Quartararo 1916 discloses a toothbrush case in which the bristles are positioned above a pad that has been saturated with a volatile sterilization liquid that gradually evaporates, thereby providing fumes which penetrate and ultimately sterilize the bristles.

U.S. Pat. No. 1,200,236 issued to Ray et. al (1916) and U.S. Pat. No. 4,884,688 issued to Hurst (1989) disclose toothbrush sanitizers having cylindrical vessels which contain a germicidal liquid in which the toothbrush bristles are immersed between uses. U.S. Pat. No. 1,451,425 issued to Hurley (1923) and U.S. Pat. No. 3,207,296 issued to Goodall (1965) also disclose sterilization containers, but Hurley and Goodall both teach devices wherein the entire toothbrush is enclosed so as to expose it to the vapors created by the evaporation of germicidal vapors emanating from saturated cloth inserts or germicidal pellets. More recently, U.S. Pat. No. 5,333,742 issued to Piedmont et al. discloses a wall-mounted toothbrush holder that uses a roll of disposable plastic bags carried on the (wall-mounted) bracket to receive the head of the toothbrush.

The prior art also contains several examples of toothbrush sanitizers that rely upon different forms of irradiation to sterilize dental objects. For example, U.S. Pat. Nos. 4,400,357 issued to Hohmann (1983) and 5,919,416 issued to Auner (1999) entail the use of microwave energy while U.S. Pat. No. 4,906,851 issued to Beasley et al. (1990), 4,973,847 issued to Lackey et. al., and 5,023,460 issued to Foster, Jr. et al. (1991) rely upon the use of ultraviolet light to sterilize toothbrush bristles. U.S. Pat. No. 4,816,648 issued to Dusbabek (1989) discloses a toothbrush sterilizer that uses an imbedded heating element which causes the sterilization cavity to be dry-heated a sufficient amount to actually sterilize the bristles of the toothbrush inserted therein.

However, these prior devices had numerous disadvantages and the fact that these toothbrush sanitizers are not in widespread use suggests that they are deficient in some way or that they were in some way unacceptable to the general consuming public. Many were bulky and cumbersome which restricted their use to a single location such as a bathroom or dressing room in the home. Similarly, many of these devices had to be mounted on a wall, counter-top, or other such vertical or horizontal surface thereby, again, restricting their use to a single location. Many of the toothbrush sanitizers disclosed by the prior art are complicated designs, have numerous parts, and are expensive to manufacture. In addition, many of these devices are difficult to clean and as such, become a potential source of bacterial growth in and of themselves.

Many of the toothbrush sanitizing devices disclosed by the prior art rely upon sophisticated sterilization methods such as exposing the toothbrush bristles to ultraviolet light or microwave energy. While such devices could effectively sterilize the bristles of a toothbrush, they would have to operate continuously between uses and thus required replacement of the ultraviolet light bulbs or microwave energy source. Further, this line of prior art devices had to be manually activated giving rise to the problem that the toothbrushes would not be properly sterilized and ready for the next use if the user forgot to switch on the device or if there was a power outage between uses. Further still, since these devices were typically enclosed, the user had no way of knowing the device was on or not unless it had an indicator installed therein. Finally, this line of devices tended to be quite expensive to manufacture.

Other toothbrush sanitizing devices disclosed by the prior art rely upon completely immersing the toothbrush bristles in a chamber or casing containing the sanitizing liquid. Besides being complex in construction, these devices are prone to leaking and cannot be easily transported should the user want to use them while traveling. Other devices disclosed by the prior art rely upon sources of sanitizing liquid that are embedded in the body of the toothbrush itself. Unfortunately, there is no way to effectively control or limit the amount of sanitizing liquid that is released and once the sanitizing liquid has been expended, the user has to purchase a replacement toothbrush or use some other method of sanitizing the toothbrush bristles. Others relied upon the insertion (into one section of an enclosed container) of a filler element which has been saturated with antiseptic or sterilizing substance so that the evaporating fumes permeate the bristles of the toothbrush enclosed therein.

While the foregoing inventions generally solved the problem of sanitizing or disinfecting a toothbrush between uses, most, if not all of them were fixed in one location. Further, all of them required the use complex mechanical devices or devices that were difficult or expensive to manufacture. Further still, many of these inventions were costly and difficult to operate. These problems were also recognized by the prior art which contains numerous examples of inventions that attempted to address the problem of sanitizing a toothbrush with a less-cumbersome and less expensive device.

U.S. Pat. No. 1,041,315 issued to Marx (1912) discloses a relatively simple protective covering for toothbrushes that has an absorbent lining. Similarly, U.S. Pat. No. 1,827,654 issued to Harper (1929) discloses a toothbrush cover made from a square of material that wraps around the toothbrush to keep it free of dirt, dust, and other such contaminants. U.S. Pat. No. 5,771,521 issued to McNamee (1998) discloses a sanitary brush cover consisting of a closable, flexible, air-permeable shroud that completely covers the bristles of a toothbrush to protect the bristles while allowing them to dry. U.S. Pat. No. 7,246,400 issued to Ryan (2007) discloses a hygienic toothbrush with a cover composed of a finely woven mesh/screen-like material which permits airflow over the bristles to dry them between uses.

The prior art contains more complex inventions as well. U.S. Pat. No. 1,451,425 issued to Hurley (1923) discloses a sterilizing holder for toothbrushes consisting of a lightweight metal container with a removable cup or container inside to hold an antiseptic gas generating substance. Similarly, U.S. Pat. No. 1,708,423 issued to MacShane (1927) discloses a portable toothbrush holder that provides for the sterilization of a toothbrush inserted therein by using vapors from the evaporation of a sterilizing liquid located in a separate chamber from that housing the toothbrush. As well, U.S. Pat. No. 3,741,378 issued to Parker (1973) discloses a protector for a toothbrush that uses the vapors from a sanitizing agent in a leak-proof compartment to sanitize the bristles of a toothbrush inserted therein. U.S. Pat. No. 4,362,241 issued to Williams (1982) discloses a device for cold disinfection of dental and medical instruments which contemplates a two-chamber design with the sanitizing agent in the form of crushable capsules in one chamber and the absorbent liner in the other.

U.S. Pat. No. 1,904,609 issued to Bleadon (1933) discloses a container for toothbrushes that is disposable, portable, and contains an absorbent lining to dry the bristles between uses. U.S. Pat. No. 2,172,600 issued to Van der Werth (1939) discloses another disposable toothbrush appliance but with a purpose of polishing or buffing the user's teeth. U.S. Pat. No. 4,384,382 issued to Diamant (1983) discloses a disposable toothbrush cover that is intended for use as a brushing device in that the exterior surface of the cover is actually used in brushing the teeth. U.S. Pat. No. 2,353,517 issued to Spanel et al. (1940) discloses a portable brush cover that can be adapted to fit over a brush such as a toothbrush but remains attached to the brush when the brush is in use. U.S. Pat. No. 5,139,142 issued to Simon (1992) also discloses a disposable toothbrush cover that it is made from laminated materials bonded together to form a sealable pouch with aeration holes to allow for aeration and drying of the bristles.

However, these devices, individually and collectively have numerous shortcomings. The absorbent lining in the device disclosed by Marx must be replaced or recharged from time to time. Similarly, the antiseptic liquid or vapor-producing agent in the devices disclosed by Hurley, MacShane, Parker, Williams, and Vermooten must be replaced or recharged from time to time. Quite significantly, the devices disclosed by Marx, Hurley, MacShane, Harper, Parker, Williams, and Vermooten are not disposable.

The devices disclosed by Marx, Simon, and Ryan are intended to dry the bristles of a toothbrush inserted therein; they do not contemplate the use of a sanitizing or sterilizing agent to disinfect the toothbrush. Similarly, the devices disclosed by Harper, Spanel, and MacNamee keep the bristles free from dirt, dust and other airborne contaminants but they do not contemplate the use of sterilizing or sanitizing agents to disinfect the toothbrush. The device disclosed by Bleadon is intended to dry the bristles of a toothbrush inserted therein while also sanitizing or sterilizing the toothbrush; however, this device, like those disclosed by Harper, Williams, and Simon do not have a leak-proof seal between the toothbrush and the cover thereby allowing the sanitizing agent to leak out of the pouch. The devices disclosed by Van der Wert, Spanel, and Diamant are intended for some purpose other than sanitizing a toothbrush and, as such do not contemplate sterilizing or sanitizing the toothbrush.

Finally, the devices disclosed by Hurley, MacShane, Parker, Williams, and Vermooten are quite complex which makes them more expensive to manufacture and consequently, more costly for the ultimate user to purchase.

In other fields, U.S. Pat. No. 2,533,829 issued to Merryweather (1950) discloses a storage bag for keeping paintbrushes and the like moist and resilient through the use of a lubricating liquid in a gas-tight flexible bag closed by a resilient cord. However, this device, like many of the toothbrush covers, does not contemplate any sanitizing or sterilizing action; further, it is not disposable. U.S. Patent Application Publication No. 2005/0160550 submitted by Reeves discloses a utility mitten that fits over a bristled brush so that the brush cover (i.e., the utility mitten) can be used to wash a car without causing damage to the finish. This device, like many of the toothbrush covers, does not contemplate any sanitizing or sterilizing action. U.S. Patent Application Publication No. 2007/0092398 submitted by McDonald discloses a gas-permeable pouch for the sterilization of medical products after the pouch is placed in a sterilization chamber. This device requires an external sterilizing and sanitizing source.

In sum, the devices disclosed by the prior art suffer numerous shortcomings and disadvantages both individually and collectively:

(1) Most of the toothbrush covers found in the prior art are intended for durable use and, as such, are not disposable.

(2) Most of the toothbrush covers found in the prior art simply cover the toothbrush when it is not in use. These devices only protect the toothbrush from dust, dirt, and the like.

(3) Most of the toothbrush covers found in the prior art do not sterilize or sanitize the toothbrush inserted therein.

(4) Most of the toothbrush covers found in the prior art simply allow the toothbrush bristles to dry when not in use.

(5) Most of the toothbrush covers found in the prior art are permanent fixtures and, as such, not portable.

While the prior art reveals some toothbrush covers that contemplate the use of sterilizing and/or sanitizing agents, these devices suffer from many of the same shortcomings as the non-sterilizing toothbrush covers as well as some disadvantages of their own:

(1) Most of the sterilizing toothbrush covers found in the prior art are intended for durable use and, as such, are not disposable.

(2) Most of the sterilizing toothbrush covers found in the prior art are permanent fixtures and, as such, not portable.

(3) Most of the sterilizing toothbrush covers found in the prior art are prone to leaking making them unsuitable for use outside of the home.

(4) Most of the sterilizing toothbrush covers found in the prior art rely upon the use vapors emanating from a sterilizing liquid or tablet placed in a chamber that is separate and apart from the chamber containing the head of the toothbrush inserted therein. As such, these devices:
(a) are complex in design and expensive to manufacture.
(b) are expensive to the consumer.
(c) require replenishment or replacement of the sterilizing agent.
(d) are not simple to operate or use.
(e) are not disposable.

### OBJECTS AND ADVANTAGES

The present invention seeks to overcome all of these shortcomings with a single device by providing an improved toothbrush sanitizer that is simple in construction, ergonomically efficient, and durable while still meeting the stated needs of the consumer for a reasonably priced and disposable toiletry item. Accordingly, the objects and advantages of the present invention are:

(1) to provide a sterilizing and disposable toothbrush cover that sterilizes the bristles of a toothbrush between uses by tightly enveloping the bristles with a material that has been treated with a sterilizing or antiseptic agent or solution.

(2) to provide a sterilizing and disposable toothbrush cover that can be simply, quickly, and easily discarded when no longer needed or wanted.

(3) to provide a sterilizing and disposable toothbrush cover that is small and compact so as to be easily transportable in a travel case such as a shaving kit, cosmetic bag, duffle bag, or the like.

(4) to provide a sterilizing and disposable toothbrush cover that will not leak.

(5) to provide a sterilizing and disposable toothbrush cover that does not have to be recharged or replenished with a sterilizing or antiseptic agent or solution.

(6) to provide a sterilizing and disposable toothbrush cover that is simple in design and constructed of inexpensive components that are easy to obtain.

(7) to provide a sterilizing and disposable toothbrush cover that is economical to manufacture and thereby, affordable to the general public.

(8) to provide a sterilizing and disposable toothbrush cover with no moving parts.

(9) to provide a sterilizing and disposable toothbrush cover that is easy to use.

Additional objects, advantages, and novel features of the invention will be set forth in part of the description which follows and will become apparent to those skilled in the art upon examination of the following specification, or will be learned through the practice of the present invention.

### DRAWINGS

### DRAWING FIGURES

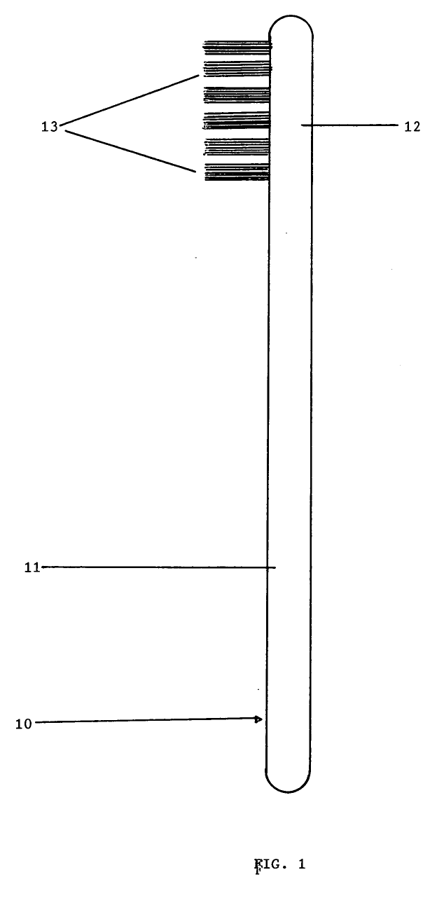

FIG. 1 is a side view of a typical toothbrush.

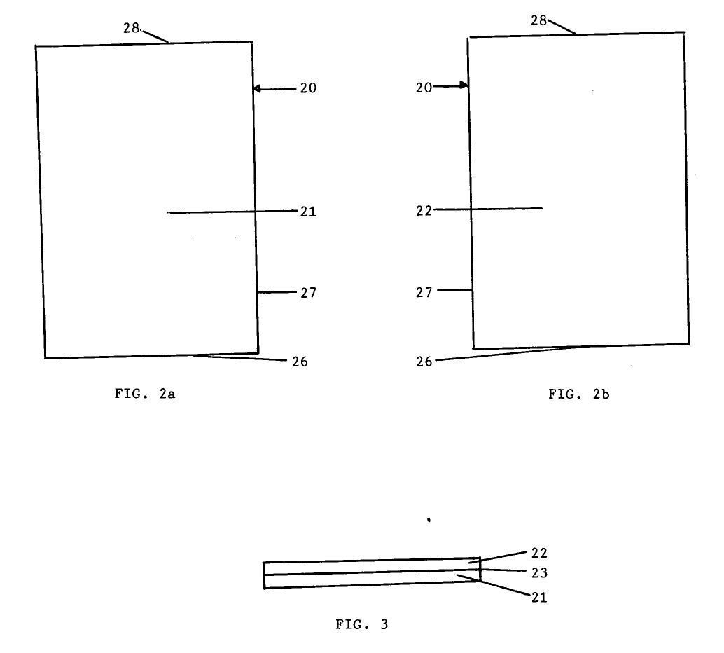

FIG. 2a is a plan view showing the inner layer of the simplest embodiment of the present invention.

FIG. 2b is a plan view showing the outer layer of the simplest embodiment of the present invention.

FIG. 3 is a cross-sectional view of the simplest embodiment of the present invention showing the inner and outer layers bonded to one another.

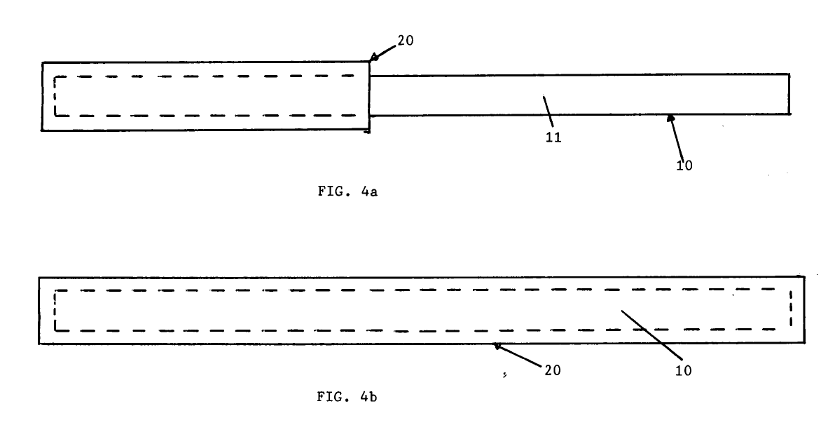

FIG. 4a is a plan view of the top of the simplest embodiment of the present invention wrapped around the head and bristles of a conventional toothbrush.

FIG. 4b is a plan view of the top of the simplest embodiment of the present invention wrapped around a conventional toothbrush.

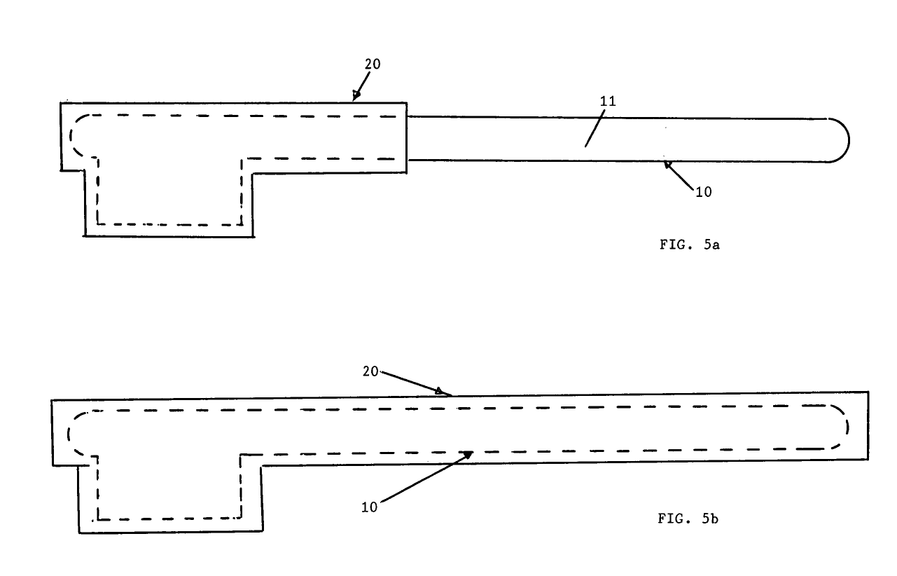

FIG. 5a is a side view of the simplest embodiment of the present invention wrapped around the head and bristles of a conventional toothbrush.

FIG. 5b is a side view of the simplest embodiment of the present invention wrapped around a conventional toothbrush.

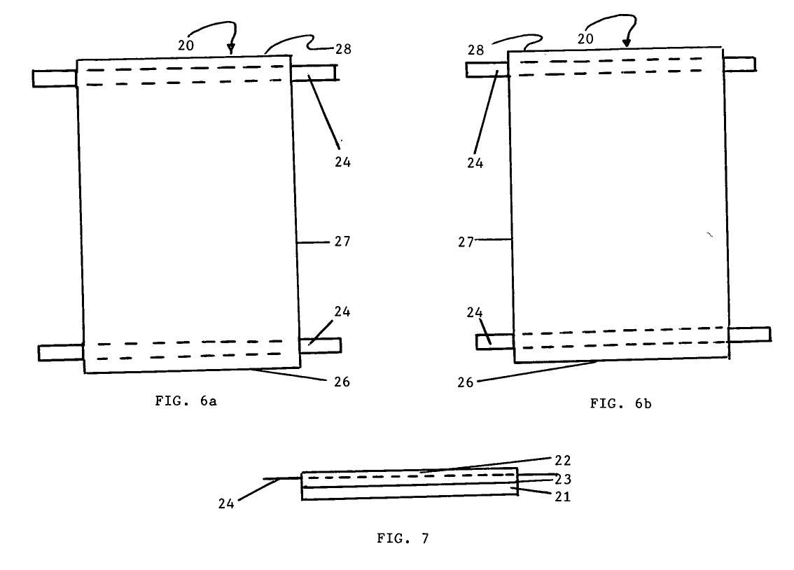

FIG. 6a is a plan view showing the inner layer of the simplest embodiment of the present invention with a tightening device.

FIG. 6b is a plan view showing the outer layer of the simplest embodiment of the present invention with a tightening device.

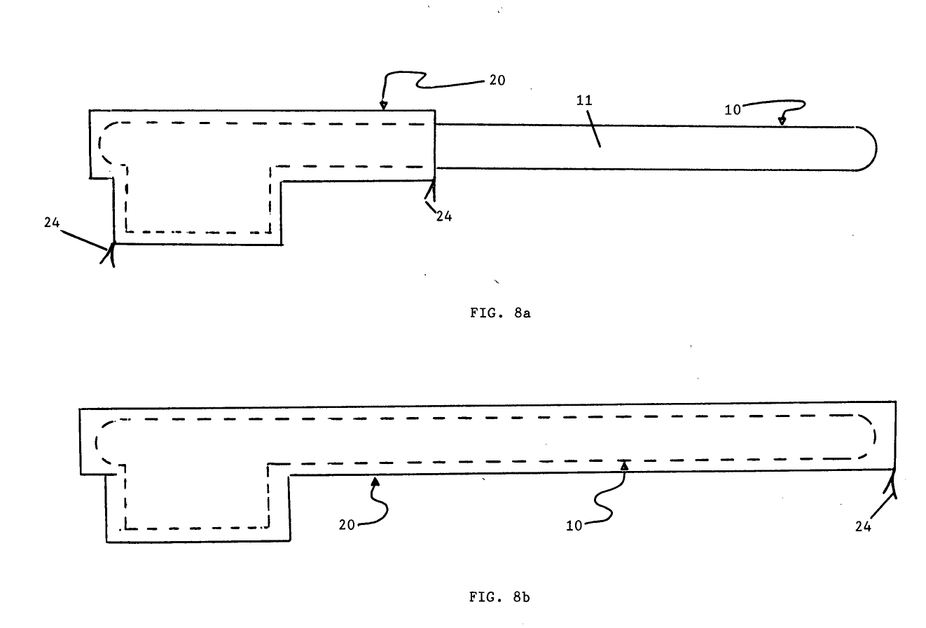

FIG. 7 is a cross-sectional view of the simplest embodiment of the present invention with tightening devices showing the inner and outer layers bonded to one another.

FIG. 8a is a side view of the simplest embodiment of the present invention wrapped around the head of a conventional toothbrush using tightening devices.

FIG. 8b is a side view of the simplest embodiment of the present invention wrapped around a conventional toothbrush using a tightening device.

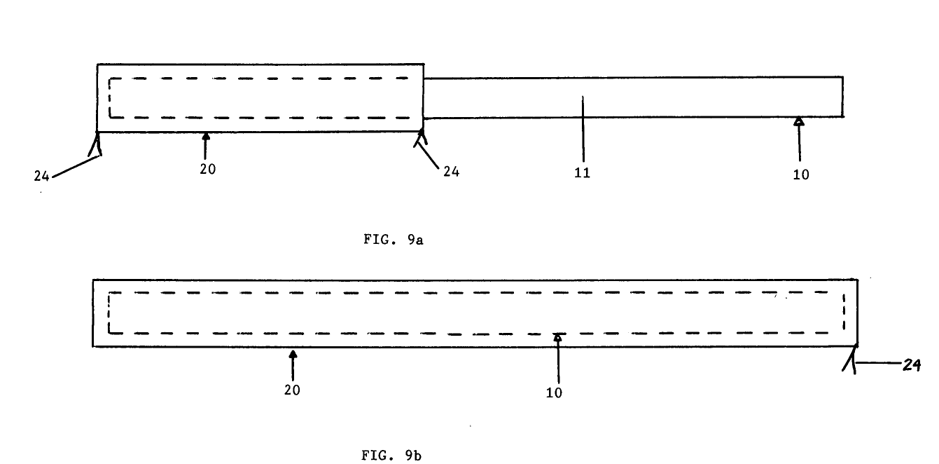

FIG. 9a is a plan view of the top of the simplest embodiment of the present invention wrapped around the head and bristles of a conventional toothbrush using tightening devices.

FIG. 9b is a plan view of the top of the simplest embodiment of the present invention wrapped around a conventional toothbrush using a tightening device.

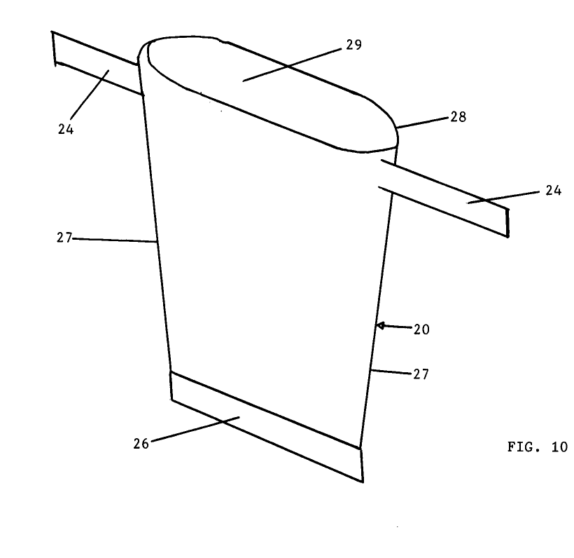

FIG. 10 is a perspective view of preferred embodiment of the present invention using tightening devices.

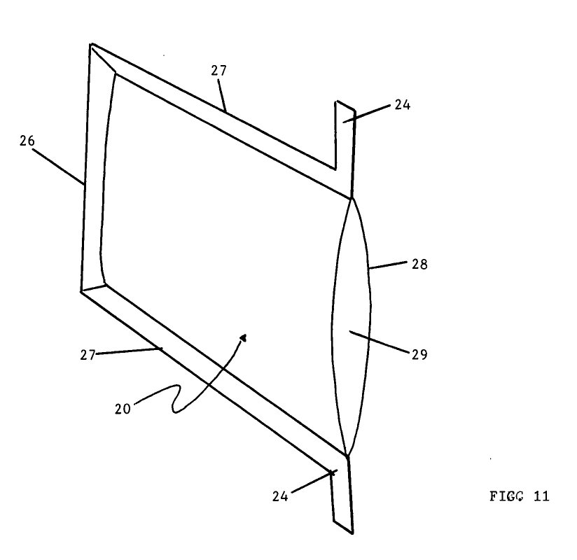

FIG. 11 is a perspective view of another version of the preferred embodiment of the present invention.

### REFERENCE NUMERALS

10—Toothbrush
11—Handle portion (“handle”)
12—Head portion (“head”)
13—Bristles
20—Shield
21—Bottom layer
22—Top layer
23—Bonding agent
24—Tightening device
26—Bottom Edge
27—Side Edge
28—Top Edge
29—Opening
30—Pouch

### DESCRIPTION OF THE INVENTION

FIG. 1 shows a side view of a typical toothbrush 10 divided into a handle potion 11 (hereinafter, referred to as the “handle” of toothbrush 10) and a head portion 12 (hereinafter referred to as the “head” of toothbrush 10) into which are inserted bristles 13. Toothbrush 10 is an oral hygiene device. The user places some type of dental abrasive paste or powder onto bristles 13 and then moves bristles 13 in a repetitive horizontal, vertical or circular motion across the surface of the teeth thereby cleaning the surface of the teeth and removing food particles from the spaces between the teeth. When not in use, toothbrush 10 is typically stored vertically with head 12 above handle 11 in some type of holder or storage rack that has been mounted on a vertical surface in the user's bathroom, dressing room, or other such place in the home or office. Often, toothbrush 10 is either stored in some type of closed plastic or metal container that lies horizontally in a cabinet drawer or on a horizontal surface near the sink where the brushing activity takes place. All too frequently, toothbrush 10 is just left on the counter. Each of the foregoing storage options will, to varying degrees, expose toothbrush 10, handle 11, head 12, and bristles 13 to contamination from germs, bacteria, viruses, disease-causing pathogens, and airborne particulate matter (such as dirt and dust) between uses.

### Description—Simplest Embodiment

The present invention addresses this contamination problem by providing the user with a simple, affordable, portable, leak-proof, and disposable sanitizing cover for toothbrush 10. In its simplest embodiment, the present invention is simply wrapped around the head 12 and bristles 13 of a conventional toothbrush 10 when the toothbrush 10 is not in use. The present invention would disinfect or sterilize the head 12 and bristles 13 wrapped inside with a sanitizing agent such as a disinfectant, antibacterial agent, anti-viral agent, or sterilizing agent while also protecting the head 12 and bristles 13 from the aforementioned types of airborne contamination. In a modified version of the simplest embodiment, the present invention is simply wrapped around the entire toothbrush 10 when the toothbrush 10 is not in use. The present invention would disinfect or sterilize the handle 11, head 12 and bristles 13 of toothbrush 10 wrapped inside while also protecting the handle 11, head 12 and bristles 13 from the aforementioned types of airborne contamination.

FIGS. 2a and 2b respectively show bottom-up and top-down plan views of the simplest embodiment of the present invention. In the simplest embodiment, the present invention is comprised of a generally rectangularly shaped shield 20 having a bottom edge 26, side edges 27, and a top edge 28. Shield 20 is comprised of a top layer 22 laminated upon and permanently bonded to bottom layer 21 by a bonding agent 23. FIG. 3 is a cross-sectional view through shield 20 that depicts bottom layer 21 bonded to top layer 22 by means of a bonding agent 23.

Bottom layer 21 is typically composed of a soft, pliable, absorbent material such as cotton cloth, gauze, cellulite, neoprene rubber, foam rubber, and the like which has been impregnated with a sanitizing agent such as an antiseptic, anti-viral, antibacterial, or disinfecting liquid, powder, or paste. Bottom layer 21 should be impregnated with a sufficient amount of the chosen sanitizing agent to ensure that the sanitizing agent will saturate or coat bristles 13 when bristles 13 come into contact with bottom layer 21. The sanitizing agent serves to kill bacteria and other disease-causing pathogens remaining on the handle 11, head 12, and bristles 13 of toothbrush 10 after it has been used.

Top layer 22 is typically composed of a soft, pliable, and leak-proof material such as aluminum foil, plastic laminate, and the like that will permanently deform and collapse upon itself when squeezed or crushed by the user. This squeezing or crushing action by the user will mold shield 20 into a protective sac or container around the periphery of the head 12 and bristles 13 of toothbrush 10. It will also prevent the sanitizing agent from leaking out of the protective sac or container.

The sanitizing agent with which bottom layer 21 has been impregnated can be a formula designed specifically for the present invention or a disinfectant, antiseptic, or anti-viral agent that is commercially available to the general public with or without a prescription. Bonding agent 23 is typically composed of some type of glue or other adhesive material that is otherwise non-reactive with the materials composing bottom layer 21, top layer 22, or the sanitizing agent with which bottom layer 21 has been impregnated.

Shield 20 is of sufficient size to completely cover and envelop head 12 and bristles 13 of a conventional toothbrush 10 once the user has crushed shield 20. While actual sizes can and will vary, a shield 20 that is approximately 2.5 inches wide and approximately 3.5 inches long would be a commercially reasonable size for the present invention. In another version of this simplest embodiment, shield 20 is of sufficient size to encase an entire toothbrush 10 including the head 12, bristles 13, and handle 11 once the user has crushed shield 20. In this version, shield 20 would typically be approximately 2.5 inches wide and approximately 9.0 inches long. Shield 20 in either version may also be fabricated in a shape other than rectangular such as, without limitation, circular, elliptical, triangular, toothbrush silhouette, and the like, but rectangular seems to be the most economic shape to manufacture and the easiest for the consumer to use.

### Operation—Simplest Embodiment

FIG. 4a is a plan view of the top of the simplest embodiment of the present invention wrapped around the head 12 and bristles 13 of a typical toothbrush 10. FIG. 4b is a plan view of the top of the simplest embodiment of the present invention wrapped around an entire toothbrush 10. FIG. 5a is a side view of the simplest embodiment of the present invention wrapped around the head 12 and bristles 13 of a typical toothbrush 10. FIG. 5b is a side view of the simplest embodiment of the present invention wrapped around an entire toothbrush 10.

When using the simplest embodiment of the present invention, the user simply wraps shield 20 around head 12 and bristles 13 of a toothbrush 10 the user desires to cover and sanitize. Alternatively, the user simply drapes shield 20 over head 12 and bristles 13 of a toothbrush 10 the user desires to cover and sanitize. In both instances, shield 20 is placed so as to completely envelop head 12 and bristles 13 and to ensure that bottom layer 21 is in direct physical contact with head 12 and bristles 13. The user then either folds or crumples shield 20 around the head 12 and bristles 13 so that shield 20 is molded into a sac or container around the head 12 and bristles 13. By folding or crumpling shield 20 in this manner, the user molds shield 20 into a sac or container that conforms to the general shape and periphery of the head 12, and bristles 13 now enclosed therein.

When the user desires to enclose the entire toothbrush 10, the user wraps shield 20 around the head 12, bristles 13 and handle 11 of a toothbrush 10 the user desires to cover and sanitize. Alternatively, the user simply drapes shield 20 over the head 12, bristles 13, and handle 11 of a toothbrush 10 the user desires to cover and sanitize. In both instances, shield 20 is placed so as to completely envelop the head 12, bristles 13, and handle 11 (i.e., the entire toothbrush 10) and to also ensure that bottom layer 21 is in direct physical contact with the handle 11, head 12 and bristles 13. The user then either folds or crumples shield 20 around the handle 11, head 12, and bristles 13 so that shield 20 is molded into a sac or container around the handle 11, head 12 and bristles 13 (i.e., the entire toothbrush 10). By folding or crumpling shield 20 in this manner, the user molds shield 20 into a sac or container that conforms to the general shape and periphery of the handle, 11, head 12, and bristles 13 (i.e., the entire toothbrush 10) now enclosed therein.

The user then folds, crimps or pinches the bottom edge 26, sides 27, and top edge 28 of shield 20 tightly and securely around handle 11 to ensure that the sanitizing agent does not leak out of the newly-formed sac or compartment. Although folding, crimping or pinching shield 20 as described above would suffice to prevent leaking, the use of a paste or dry powder to impregnate lower layer 21 would further guarantee that the sanitizing agent does not leak out. Further, the use of a paste or dry powder to impregnate bottom layer 21 would tend to dry bristles 13 and extend their useful life.

Where the entire toothbrush 10 is enclosed by the present invention, the user folds, crimps, or pinches the bottom edge 26, sides 27, and top edge 28 of shield 20 tightly and securely back upon itself to completely close shield 20 upon itself and thereby ensure that the sanitizing agent does not leak out of the newly-formed sac or compartment. Although folding, crimping or pinching shield 20 as described above would suffice to prevent leaking, the use of a paste or dry powder to impregnate lower layer 21 would further guarantee that the sanitizing agent does not leak out. Further, the use of a paste or dry powder to impregnate bottom layer 21 would tend to dry bristles 13 and extend their useful life.

While the head 12 and bristles 13 of toothbrush 10 or the entire toothbrush 10 (as the case may be) are covered by shield 20 as described above, they are subjected to the sanitizing effect of the sanitizing agent applied to inner layer 21. The user may wish to squeeze shield 20 to forcibly release more of the sanitizing agent from bottom layer 21 onto the head 12 and bristles 13 of toothbrush 10 or the entire toothbrush 10 (as the case may be). The head 12 and bristles 13 of toothbrush 10 or the entire toothbrush 10 (as the case may be) are also protected from contamination by airborne contaminants such as dust, dirt, and the like. The covered toothbrush 10 can now be stored or transported as the user desires without any concern about the sanitizing agent leaking onto a counter top, into a storage drawer, or into a suitcase, overnight bag, shaving kit, or the like. When the user is ready to use the covered toothbrush 10 again, he or she simply peels back the bottom edge 26, sides 27, and top edge 28 of shield 20, slips shield 20 off the head 12 and bristles 13 of toothbrush 10 or the entire toothbrush 10 as the case may be, and then disposes of the used shield 20 (i.e., the present invention) in a proper receptacle.

### Description—Simplest Embodiment with Tightening Devices

Top layer 22 of shield 20 may be composed of a material that does not deform and collapse upon itself when crumpled or crushed by the user. The leak-proof feature of the present invention would be provided through some means of tightening shield 20 around toothbrush 10 or handle 11 so that the sanitizing agent does not leak out of the resultant pouch or container. The means contemplated by this modification to the simplest embodiment involves the use of a tightening device 24 imbedded in or externally attached to shield 20.

Tightening device 24 may be any means that draw the top edge 28, sides 27, and bottom edge 26 of shield 20 tightly and securely around handle 11 so as to form a leak-proof pouch or container around the head 12 and bristles 13. When the present invention envelops toothbrush 10 in its entirety, tightening device 24 may be any means by which the user can tightly and securely draw together and close the top edge 28, sides 27, and bottom edge 26 of shield 20 so as to form a leak-proof sac or container around toothbrush 10. As such, tightening device 24 can be composed of an elastic band slipped over shield 20 once shield 20 has been wrapped around or draped over head 12 and bristles 13. Tightening device 24 can also be composed of natural or artificial fiber purse strings imbedded in or attached externally to shield 20. Tightening device 24 can also be composed of semi-rigid metal wires (similar to a pipe-cleaner or a grocery store “twist-it”) imbedded in or attached externally to shield 20. Finally, tightening device 24 can be composed of any other such means of tightening and securing shield 20 to handle 11 so as to form a leak-proof seal between shield 20 and handle 11. When enclosing the entire toothbrush is desired, tightening device 24 can be composed of, without limitation, an elastic band slipped over shield 20 once shield 20 has been wrapped around or draped over head 12 and bristles 13, natural or artificial fiber purse strings imbedded in or attached externally to shield 20, semi-rigid metal wires (similar to a pipe-cleaner or a grocery store “twist-it”) imbedded in or attached externally to shield 20, or other such means of closing the top edge 28, sides 27, and bottom edge 26 of shield 20 so as to form a leak-proof sac or container surrounding toothbrush 10.

While having tightening devices 24 near the top edge 28 and the bottom edge 26 of shield 20 affords the user the greatest protection against possible leakage, using a single tightening device 24 near either the top edge 28 or the bottom edge 26 of shield 20 is possible as well. When using the single tightening device 24 option, the user simply wraps the edge with the tightening device 24 around and over the edge without the tightening device after shield 20 has been wrapped around or draped over the head 12 and bristles 13 or the entire toothbrush 10 as the case may be.

In sum, tightening device 24 tightens shield 20 around handle 11 and secures shield 20 thereto so as to prevent shield 20 from slipping off handle 11 while also preventing any the sanitizing agent with which bottom layer 21 is impregnated from leaking out onto handle 11 or running down handle 11. When shield 20 encases the entire toothbrush 10, tightening device 24 tightly and securely closes the top edge 28, sides 27, and bottom edge 26 of shield 20 around the entire toothbrush 10 so as to create a leak-proof sac or container and thereby sanitizing agent with which bottom layer 21 is impregnated from leaking out of the sac or container now encasing toothbrush 10.

FIGS. 6a and 6b respectively show bottom-up and top-down plan views of the simplest embodiment of the present invention but now modified with tightening devices 24 imbedded in top layer 22 near the top edge 28 and the bottom edge 26 of shield 20. Tightening device 24 can also be externally attached to top layer 22 of shield 20.

FIG. 7 is a cross-sectional view showing bottom layer 21 bonded by a bonding agent 23 to top layer 22 of shield 20 (as found in the simplest embodiment of the present invention) but modified with a tightening device 24 embedded in the top layer 22. FIG. 8a is a side view of the simplest embodiment of the present invention wrapped around the head 12 and bristles 13 of a conventional toothbrush 10 using a two tightening devices 24. FIG. 8b is a side view of the simplest embodiment of the present invention wrapped around an entire conventional toothbrush 10 using a single tightening device 24. FIG. 9a is a plan view of the simplest embodiment of the present invention wrapped around the head 12 and bristles 13 of a conventional toothbrush 10 using a two tightening devices 24. FIG. 9b is a side view of the simplest embodiment of the present invention wrapped around an entire conventional toothbrush 10 using a single tightening device 24.

### Operation—Simplest Embodiment with Tightening Device

In using the single-fastening device 24 option, the user drapes shield 20 over the head 12 and the bristles 13 of a toothbrush 10 the user desires to cover and sanitize so as to envelop the head 12 and the bristles 13 and to ensure that bottom layer 21 is in direct physical contact with head 12 and bristles 13. The user would then tuck the loose folds of shield 20 into themselves while tightly rolling shield 20 around the head 12 and the bristles 13. The user then tightens shield 20 around itself and the handle 11 using the single tightening device 24 thereby securing shield 20 to handle 11 so as to form a leak-proof sac or container around the head 12 and bristles 13. If the user wishes to envelop the entire toothbrush 10, the user simply wraps shield 20 around toothbrush 10 or drapes shield 20 over toothbrush 10 so as to completely envelop toothbrush 10 and ensure that bottom layer 21 is in direct physical contact with handle 11, head 12, and bristles 13. The user then tucks the loose folds of shield 20 into themselves while tightly rolling shield 20 around toothbrush 10. The user then tightly and securely closes the shield 20 around itself using the single tightening device 24 so as to form a leak-proof sac or container now enveloping and containing toothbrush 10.

If tightening device 24 is an elastic band, the user would simply slip tightening device 24 over shield 20 and head 12 and bristles 13 once shield 20 has been wrapped around or draped over and then rolled around head 12 and bristles 13 or the entire toothbrush 10. If tightening device 24 is a set of natural or artificial fiber purse strings imbedded in or attached externally to shield 20, the user would pull on the opposite ends of tightening device 24 once shield 20 has been wrapped around or draped over and rolled around head 12 and bristles 13 or the entire toothbrush 10 and then tie the purse string(s) in a knot. If tightening device 24 is a semi-rigid metal wire (similar to a pipe-cleaner or a grocery store “twist-it”) imbedded in or attached externally to shield 20, the user would twist tightening device 24 once shield 20 has been wrapped around or draped over and rolled around head 12 and bristles 13 or the entire toothbrush 10.

In using the two-fastening device 24 option, the user would roll shield 20 around the head 12 and bristles 13 and then tighten shield 20 in two locations as shown in FIGS. 8a and 9a which would make the entire arrangement less bulky. On the other hand, the user could drape shield 20 over the head 12 and bristles 13 as discussed in using the single-fastening device 24 option and then proceed to tighten the tightening device 24 near the bottom edge 26 of shield 20 first. Once the tightening device 24 near the bottom edge has been tightened, the user would roll shield 20 around the head 12 and bristles 13 and then proceed to tighten the tightening device 24 near the top edge 28 of shield 20.

In all cases, the user would slip, pull, or twist tightening device 24 tightly enough so as to prevent any of the sanitizing agent with which the bottom layer 21 is impregnated from leaking out onto handle 11 and running down handle 11. When shield 20 encases the entire toothbrush 10, the user would slip, pull, or twist tightening device 24 tightly enough so that tightening device 24 closes shield 20 upon itself tightly enough so as to prevent any of the sanitizing agent with which bottom layer 21 is impregnated from leaking out of the sac created by closing shield 20 upon itself.

While the head 12 and bristles 13 of toothbrush 10 or the entire toothbrush 10 (as the case may be) are covered by shield 20 as described above, they are subjected to the sanitizing effect of the sanitizing agent applied to inner layer 21. The user may wish to squeeze shield 20 to forcibly release more of the sanitizing agent from bottom layer 21 onto the head 12 and bristles 13 of toothbrush 10 or the entire toothbrush 10 (as the case may be). The head 12 and bristles 13 of toothbrush 10 or the entire toothbrush 10 (as the case may be) are also protected from contamination by airborne contaminants such as dust, dirt, and the like. The covered toothbrush 10 can now be stored or transported as the user desires without any concern about the sanitizing agent leaking onto a counter top, into a storage drawer, or into a suitcase, overnight bag, shaving kit, or the like. When the user is ready to use the covered toothbrush 10 again, he or she simply peels back the tightening devices 24, bottom edge 26, sides 27, and top edge 28 of shield 20, slips shield 20 off the head 12 and bristles 13 of toothbrush 10 or the entire toothbrush 10 as the case may be, and then disposes of the used shield 20 (i.e., the present invention) in a proper receptacle.

### Description—Preferred Embodiment

FIGS. 10 and 11 show perspective views of the preferred embodiment of the present invention wherein two generally rectangularly-shaped shields 20 are joined together so that the bottom layers 21 face each other. The bottom edge 26 and sides 27 of each shield 20 are fused or otherwise joined together leaving an opening 29 between the top edges 28 and thereby forming a pouch 30 that is large enough to permit the user to insert the head 12 and bristles 13 of typical toothbrush 10 therein. In another version of the preferred embodiment, pouch 30 is large enough to accommodate the entire toothbrush 10.

The bottom layer 21 of shields 20 comprising the preferred embodiment, is typically composed of a soft, pliable, absorbent material such as cotton cloth, gauze, cellulite, neoprene rubber, foam rubber, and the like which has been impregnated with a sanitizing agent such as an antiseptic, anti-viral, or disinfecting liquid, powder, or paste. Bottom layer 21 should be impregnated with a sufficient amount of the chosen sanitizing agent to ensure that the sanitizing agent will saturate or coat bristles 13 when bristles 13 come into contact with bottom layer 21. The sanitizing agent serves to kill bacteria and other disease-causing pathogens remaining on the handle 11, head 12, and bristles 13 of toothbrush 10 after it has been used.

The top layer 22 of the shields 20 comprising the preferred embodiment is typically composed of a soft, pliable, and leak-proof material such as aluminum foil, plastic laminate, and the like that will permanently deform and collapse upon itself when squeezed or crushed by the user. This squeezing or crushing action by the user will mold shield 20 into a protective pouch or container around the periphery of the head 12 and bristles 13 of toothbrush 10. It will also prevent the sanitizing agent from leaking out of the protective pouch or container.

The sanitizing agent with which bottom layer 21 has been impregnated can be a formula designed specifically for the present invention or a disinfectant, antiseptic, or anti-viral agent that is commercially available to the general public with or without a prescription. Bonding agent 23 is typically composed of some type of glue or other adhesive material that is otherwise non-reactive with the materials composing bottom layer 21, top layer 22, or the sanitizing agent with which bottom layer 21 has been impregnated.

Pouch 30 is of sufficient size to completely cover the head 12 and bristles 13 of a conventional toothbrush 10 inserted therein. While actual sizes can and will vary, a pouch 30 that is approximately 2.5 inches wide and approximately 3.5 inches long would be a commercially reasonable size for the present invention. In another version of this embodiment, pouch 30 is of sufficient size to completely encase the entire toothbrush 10, including the head 12, bristles 13, and handle 11. In this version, pouch 30 would typically be approximately 2.5 inches wide and approximately 9.0 inches long. Pouch 30 may also be fabricated in a shapes other than rectangular such as, without limitation, circular, elliptical, triangular, toothbrush silhouette, and the like, but rectangular seems to be the most economic shape to manufacture and the easiest for the consumer to use.

In the event that top layer 22 of pouch 30 is composed of a material that does not deform and collapse upon itself when crumpled or crushed by the user, the leak-proof feature of the present invention is provided through some means of tightening pouch 30 around toothbrush 10 or handle 11 so that the sanitizing agent with which bottom layer 21 has been impregnated does not leak out of pouch 30. The means contemplated by this modification to the preferred embodiment involves the use of a tightening device 24 imbedded in or externally attached to the pouch 30 near or along the top edge 28.

Tightening device 24 may be any means that draws the top edge 28 of pouch 30 tightly and securely around handle 11 so as to form a leak-proof seal between pouch 30 and handle 11. In the case of enveloping toothbrush 10 in its entirety, tightening device 24 may be any means that tightly and securely draws and closes the top edge 28 of pouch 30 so as to form a leak-proof container around the toothbrush 10 enclosed therein. As such, tightening device 24 can be composed of, without limitation, an elastic band slipped over pouch 30 once the head 12 and bristles 13 have been inserted therein, natural or artificial fiber purse strings imbedded in or attached externally to the top layer 22 of pouch 30, semi-rigid metal wires (similar to a pipe-cleaner or a grocery store “twist-it”) imbedded in or attached externally to the top layer 22 of pouch 30, or other such means of tightening and securing pouch 30 to handle 11 so as to form a leak-proof seal between the pouch 30 and handle 11. When enclosing the entire toothbrush is desired, tightening device 24 can be composed of, without limitation, an elastic band slipped over pouch 30 once the toothbrush 10 has been inserted therein, natural or artificial fiber purse strings imbedded in or attached externally to pouch 30, semi-rigid metal wires (similar to a pipe-cleaner or a grocery store “twist-it”) imbedded in or attached externally to pouch 30, or other such means of closing the top edge 28 of pouch 30 so as to form a leak-proof container surrounding toothbrush 10.

### Operation—Preferred Embodiment

In operation, the user simply inserts the head 12 and bristles 13 of a recently used toothbrush 10 or, in some cases, the entire toothbrush 10, into pouch 30 so as to completely envelop head 12 and bristles 13 or toothbrush 10 and to ensure that bottom layer 21 is in direct physical contact with head 12 and bristles 13 or the entire toothbrush 10. The user then either folds pouch 30 around head 12 and bristles 13 or toothbrush 10 by crumpling pouch 30 around head 12 and bristles 13 or toothbrush 10 so that pouch 30 is molded around and generally conforms to the periphery of head 12 and bristles 13 or toothbrush 10 as the case may be.

The user then folds, crimps or pinches the top edge 28 of pouch 30 tightly and securely around handle 11 to ensure that the sanitizing which bottom layer 21 has been impregnated does not leak out of the now-closed pouch 30. When enclosing the entire toothbrush 10 is desired, the user folds, crimps or pinches the top edge 28 of pouch 30 tightly and securely back on itself or against itself so that pouch 30 is now closed and the sanitizing agent with which bottom layer 21 has been impregnated cannot leak out. Although crimping or pinching pouch 30 as described above would suffice to prevent leaking, the use of a paste or dry powder to impregnate bottom layer 21 would further guarantee that the sanitizing agent does not leak out. Further, the use of a paste or dry powder to impregnate bottom layer 21 would tend to dry bristles 13 and extend their useful life.

In using the preferred embodiment equipped with a tightening device 24, the user would simply insert the head 12 and bristles 13 or the entire toothbrush 10 into the opening 29 in pouch 30 and then draw tightening device 24 so as to tightly and securely close pouch 30 around handle 11 or the entire toothbrush 10.

If tightening device 24 is an elastic band, the user would simply slip tightening device 24 over pouch 30 once the head 12 and bristles 13 or the toothbrush 10 had been inserted therein. Similarly, if tightening device 24 is a set of natural or artificial fiber purse strings imbedded in or attached externally to purse 30, the user would simply pull on the opposite ends of tightening device 24 once the head 12 and bristles 13 or the toothbrush 10 had been inserted into pouch 30 and then tie off the purse string in a knot. If tightening device 24 is a semi-rigid metal wire (similar to a pipe-cleaner or a grocery store “twist-it”) imbedded in or attached externally to pouch 30, the user would twist tightening device 24 once the head 12 and bristles 13 or toothbrush 10 had been inserted therein.

In all cases, the user would slip, pull, or twist tightening device 24 tightly enough so as to prevent any of the sanitizing with which bottom layer 21 is impregnated from leaking out onto handle 11 and running down handle 11. When pouch 30 encases the entire toothbrush 10, the user would slip, pull, or twist tightening device 24 tightly enough so that tightening device 24 closes pouch 30 upon itself tightly enough so as to prevent any of sanitizing agent with which bottom layer 21 is impregnated from leaking out.

While the head 12 and bristles 13 of toothbrush 10 or the entire toothbrush 10 (as the case may be) are covered by pouch 30 as described above, they are subjected to the sanitizing effect of the sanitizing agent applied to inner layer 21. The user may wish to squeeze pouch 30 to forcibly release more of the sanitizing agent from bottom layer 21 onto the head 12 and bristles 13 of toothbrush 10 or the entire toothbrush 10 (as the case may be). The head 12 and bristles 13 of toothbrush 10 or the entire toothbrush 10 (as the case may be) are also protected from contamination by airborne contaminants such as dust, dirt, and the like. The covered toothbrush 10 can now be stored or transported as the user desires without any concern about the sanitizing agent leaking onto a counter top, into a storage drawer, or into a suitcase, overnight bag, shaving kit, or the like. When the user is ready to use the covered toothbrush 10 again, he or she simply peels back the top edge 28 or the tightening devices 24 (as the case may be) of pouch 30, slips pouch 30 off the head 12 and bristles 13 of toothbrush 10 or the entire toothbrush 10 as the case may be, and then disposes of the used pouch 30 (i.e., the preferred embodiment of the present invention) in a proper receptacle.

### CONCLUSIONS, RAMIFICATIONS, AND SCOPE.

The foregoing paragraphs describe an invention that has successively overcome the shortcomings experienced by practitioners of the prior art. The present invention provides the consumer with a simple device and method of protecting his or her toothbrush from numerous types of contamination between uses. The present invention will be easy and economical to manufacture which translates to lower costs to the consuming public. The present invention is portable, leak-proof and disposable making it a “must-have” item for a consuming public that is continually on the go. The present invention addresses a serious health risk that makes it all the more desirable to a health-conscious consuming public. In short, the present invention has built upon the contributions provided by the prior art and filled in the gaps with innovative solutions that give the consumer an economical, portable, and disposable solution to sterilizing a toothbrush between uses.

The present invention can be conveniently packaged so that the user can carry it on his or her person, in a suitcase or overnight bag, or in a shaving kit or cosmetics bag. This type of convenient packaging would also lend itself to keeping the present invention on hand in one's desk at work or in the glove compartment of one's car. Because it is economical, portable, and disposable, the present invention will provide the user with a welcome alternative to the limited selection of toothbrush sanitizing devices currently available commercially for purchase by the public.

### SUMMARY

The present invention has overcome the shortcomings inherent in the prior art by providing an improved toothbrush sanitizing device that is simple in construction, ergonomically efficient, and durable while still meeting the stated needs of the consumer for a reasonably priced and disposable toiletry item. The present invention addresses each of the aforementioned shortcomings by:

providing a sterilizing and disposable toothbrush cover that sterilizes the bristles of a toothbrush between uses by tightly enveloping the bristles with a material that has been treated with a sterilizing or antiseptic agent or solution.

providing a sterilizing and disposable toothbrush cover that can be simply, quickly, and easily discarded when no longer needed or wanted.

providing a sterilizing disposable toothbrush cover that is small and compact so as to be easily transportable in a travel case such as a shaving kit, cosmetic bag, duffel bag, or the like.

providing a sterilizing and disposable toothbrush cover that will allow the sterilizing agent to leak out.

providing a sterilizing and disposable toothbrush cover that does not have to be recharged or replenished with sterilizing or sanitizing liquid.

providing a sterilizing and disposable toothbrush cover that is simple in design and constructed of inexpensive components that are easy to obtain.

providing a sterilizing and disposable toothbrush cover that is economical to manufacture and thereby, affordable to the general public.

providing a sterilizing and disposable toothbrush cover with no moving parts that will ultimately fail.

providing a sterilizing and disposable toothbrush cover that is easy to use.

What is claimed is:

1. A disposable sanitizing toothbrush cover that is both portable and leak-proof comprising a disposable sanitizing shield having a bottom layer composed of a soft, pliable, absorbent material that has been impregnated with a sanitizing agent and a top layer composed of a soft, pliable, and leak-proof material that will permanently deform and collapse upon itself when squeezed or crushed, said bottom layer permanently attached to said top layer by a bonding agent and said shield being large enough and shaped so as to permit wrapping or draping said shield around bristles attached to a conventional toothbrush.

2. The disposable sanitizing toothbrush cover according to claim 1 wherein said shield has a rectangular shape.

3. The disposable sanitizing toothbrush cover according to claim 1 wherein said shield has a closed curvilinear shape.

4. The disposable sanitizing toothbrush cover according to claim 1 wherein said shield is large enough and shaped so as to permit wrapping or draping said shield around a conventional toothbrush.

5. The disposable sanitizing toothbrush cover according to claim 4 wherein said shield has a rectangular shape.

6. The disposable sanitizing toothbrush cover according to claim 4 wherein said shield has a closed curvilinear shape.

7. A disposable sanitizing toothbrush cover that is both portable and leak-proof comprising:
a. a disposable sanitizing shield having a bottom layer composed of a soft, pliable, absorbent material that has been impregnated with a sanitizing agent and a top layer composed of a soft, pliable, and leak-proof material, said bottom layer permanently attached to said top layer by a bonding agent and said shield being large enough and shaped so as to permit wrapping or draping said shield around bristles attached to a head of a conventional toothbrush, and
b. a means for tightening said shield around said head of said toothbrush and forming a closed, leak-proof sac around said head of said toothbrush.

8. The disposable sanitizing toothbrush cover according to claim 7 wherein said means is an elastic band that is slipped over said shield and said head of said toothbrush.

9. The disposable sanitizing toothbrush cover according to claim 7 wherein said means is a drawable natural or artificial fiber purse string embedded in or attached to said top layer of said shield.

10. The disposable sanitizing toothbrush cover according to claim 7 wherein said means is a semi-rigid wire embedded in or externally attached to said top layer of said shield.

11. The disposable sanitizing toothbrush cover according to claim 7 wherein said shield has a rectangular shape.

12. The disposable sanitizing toothbrush cover according to claim 11 wherein said means is an elastic band that is slipped over said shield and said head of said toothbrush.

13. The disposable sanitizing toothbrush cover according to claim 11 wherein said means is a drawable natural or artificial fiber purse string embedded in or attached to said top layer of said shield.

14. The disposable sanitizing toothbrush cover according to claim 11 wherein said means is a semi-rigid wire embedded in or externally attached to said top layer of said shield.

15. A disposable sanitizing toothbrush cover that is both portable and leak-proof comprising two generally rectangular-shaped shields fused or otherwise permanently joined together on three sides so as to form a pouch that is large enough to accommodate bristles attached to a head of a conventional toothbrush inserted therein, each of said shields having a bottom layer composed of a soft, pliable, absorbent material that has been impregnated with a sanitizing agent and a top layer composed of a soft, pliable, and leak-proof material that will permanently deform and collapse upon itself when squeezed or crushed, said bottom layer permanently attached to said top layer by a bonding agent.

16. A disposable sanitizing toothbrush cover that is both portable and leak-proof comprising:
a. two generally rectangular-shaped shields fused or otherwise permanently joined together on three sides so as to form a pouch that is large enough to accommodate bristles attached to a head of a conventional toothbrush inserted therein, each of said shields having a bottom layer composed of a soft, pliable, absorbent material that has been impregnated with a sanitizing agent and a top layer composed of a soft, pliable, and leak-proof material, said bottom layer permanently attached to said top layer by a bonding agent, and
b. a means for tightening said pouch around said head of said toothbrush and forming a closed, leak-proof sac around said head of said toothbrush.

17. The disposable sanitizing toothbrush cover according to claim 16 wherein said means is an elastic band that is slipped over said shield and said head of said toothbrush.

18. The disposable sanitizing toothbrush cover according to claim 16 wherein said means is a drawable natural or artificial fiber purse string embedded in or attached to said top layer of said shield.

19. The disposable sanitizing toothbrush cover according to claim 16 wherein said means is a semi-rigid wire embedded in or externally attached to said top layer of said shield.

20. The disposable sanitizing toothbrush cover according to claim 16 wherein said pouch is large enough to entirely enclose a conventional toothbrush inserted therein.

21. The disposable sanitizing toothbrush cover according to claim 20 wherein said means is an elastic band that is slipped over said shield and said head of said toothbrush.

22. The disposable sanitizing toothbrush cover according to claim 20 wherein said means is a drawable natural or artificial fiber purse string embedded in or attached to said top layer of said shield.

23. The disposable sanitizing toothbrush cover according to claim 20 wherein said means is a semi-rigid wire embedded in or externally attached to said top layer of said shield.

24. A method for covering and sanitizing a toothbrush comprising the steps of:
a. draping or wrapping a disposable sanitizing shield having a bottom layer composed of a soft, pliable, absorbent material that has been impregnated with a sanitizing agent and a top layer composed of a soft, pliable, and leak-proof material that will permanently deform and collapse upon itself when squeezed or crushed, said bottom layer permanently attached to said top layer by a bonding agent and said shield being large enough and shaped so as to permit wrapping or draping said shield around bristles attached to a handle of a conventional toothbrush,
b. squeezing or crushing said shield so as to collapse and permanently deform said shield, and
c. molding said shield around said bristles and said handle of said toothbrush so as to create a leak-proof sac or container around said bristles and said handle.

25. The method for covering and sanitizing a toothbrush according to claim 24 wherein said shield is draped over or wrapped around a conventional toothbrush so as to completely enclose said toothbrush and wherein said shield is molded around all of said toothbrush.

26. The method for covering and sanitizing a toothbrush according to claim 24 comprising the additional step of squeezing said shield so as to forcibly release said sanitizing agent onto said bristles.

27. The method for covering and sanitizing a toothbrush according to claim 25 comprising the additional step of squeezing said shield so as to forcibly release said sanitizing agent onto said bristles.

28. A method for covering and sanitizing a toothbrush comprising the steps of:
a. draping or wrapping a disposable sanitizing shield having a bottom layer composed of a soft, pliable, absorbent material that has been impregnated with a sanitizing agent and a top layer composed of a soft, pliable, and leak-proof material, said bottom layer permanently attached to said top layer by a bonding agent and said shield being large enough and shaped so as to permit wrapping or draping said shield around bristles attached to a handle of a conventional toothbrush,
b. folding said shield's sides and edges around said bristles and said handle of said toothbrush so as to create a sac or container around said bristles and said handle, and
c. tightening said shield around said bristles and said handle of said toothbrush by means of tightening devices embedded in or externally attached to said top layer of said shield so as to prevent said sanitizing agent from leaking out of said shield and onto said handle of said toothbrush.

29. The method for covering and sanitizing a toothbrush according to claim 28 wherein said shield is draped over or wrapped around a conventional toothbrush so as to completely enclose said toothbrush and wherein said shield is molded around all of said toothbrush.

30. The method for covering and sanitizing a toothbrush according to claim 28 comprising the additional step of squeezing said shield so as to forcibly release said sanitizing agent onto said bristles.

31. The method for covering and sanitizing a toothbrush according to claim 29 comprising the additional step of squeezing said shield so as to forcibly release said sanitizing agent onto said bristles.

32. A method for covering and sanitizing a toothbrush comprising the steps of:
a. inserting bristles attached to a conventional toothbrush into a disposable toothbrush cover comprised of two generally rectangular-shaped shields fused or otherwise permanently joined together on three sides so as to form a pouch that is large enough to accommodate said bristles attached to a head of a conventional toothbrush, each of said shields having a bottom layer composed of a soft, pliable, absorbent material that has been impregnated with a sanitizing agent and a top layer composed of a soft, pliable, and leak-proof material that will permanently deform and collapse upon itself when squeezed or crushed,, said bottom layer permanently attached to said top layer by a bonding agent
b. squeezing or crushing said pouch so as to collapse and permanently deform said pouch, and
c. molding said pouch around said bristles and said handle of said toothbrush so as to create a leak-proof sac or container around said bristles and said handle.

33. The method for covering and sanitizing a toothbrush according to claim 32 wherein an entire toothbrush is inserted into said pouch and wherein said pouch is molded around all of said toothbrush.

34. The method for covering and sanitizing a toothbrush according to claim 32 comprising the additional step of squeezing said pouch so as to forcibly release said sanitizing agent onto said bristles.

35. A method for covering and sanitizing a toothbrush comprising the steps of:
a. inserting bristles attached to a conventional toothbrush into a disposable toothbrush cover comprised of two generally rectangular-shaped shields fused or otherwise permanently joined together on three sides so as to form a pouch that is large enough to accommodate said bristles attached to a head of a conventional toothbrush, each of said shields having a bottom layer composed of a soft, pliable, absorbent material that has been impregnated with a sanitizing agent and a top layer composed of a soft, pliable, and leak-proof material, said bottom layer permanently attached to said top layer by a bonding agent
b. tightening said pouch's top edge around said bristles and said handle of said toothbrush by means of tightening devices embedded in or externally attached to said top layer of said pouch so as to prevent said sanitizing agent from leaking out of said pouch and onto said handle of said toothbrush.

36. The method for covering and sanitizing a toothbrush according to claim 35 wherein an entire toothbrush is inserted into said pouch and wherein said pouch is molded around all of said toothbrush.

37. The method for covering and sanitizing a toothbrush according to claim 35 comprising the additional step of squeezing said pouch so as to forcibly release said sanitizing agent onto said bristles.
